(19) United States
(12) Patent Application Publication
(10) Pub. No.: US 2005/0199245 A1
(43) Pub. Date: Sep. 15, 2005

(54) Nose airway for aromatherapy and detecting airborne pathogens

(76) Inventor: H. George Brennan, Newport Beach, CA (US)

Correspondence Address: Michael G. Petit, P.O. Box 91929, Santa Barbara, CA, 93190-1929, US

(21) Appl. No.: 11/115,008

(22) Filed: Apr. 25, 2005

Related U.S. Application Data
(63) Continuation-in-part of application No. 10/008,870, filed on Nov. 2, 2001.

(51) Int. Cl. A62B 7/00; A61M 15/08

(52) U.S. Cl. 128/207.18

(57) ABSTRACT

A nasal airway for insertion in the nose for the delivery of volatile compositions into air entering the nose or, in a second embodiment, for the detection of targeted airborne pathogenic organisms present in the respirated airstream. The volatile compositions include aromatherapeutic compositions and scents.

This application is a continuation-in-part of copending U.S. application Ser. No. 10/008,870, filed Nov. 2, 2001.

### BACKGROUND OF THE INVENTION

1. Field of the Invention

This invention relates to a device adapted for temporary insertion within the nose for detecting the presence of airborne pathogens and the delivery of aromatherapeutic compositions.

2. Description of the Prior Art

Various forms of nasal airway devices are known in the art. Examples of such devices are disclosed in the following U.S. Pat. Nos.: 851,048; 513,458, 4,414,977; 4,201,207; 2,515,756; 1,255,578; 1,481,581; 1,597,331; 1,672,591; 1,709,740; 1,135,675; 1,014,076; 1,014,758; 1,077,574 and British Patent GB0768488; British Patent 4,148; Italian Patent IT0490828 and French Patent 7807130. While most of these devices provide a means for dilating the nostrils and maintaining an airway permitting improved air flow therethrough, each has limitations producing less than optimum air flow or discomfort which prevents the device from being used for a prolonged period of time. Many devices are unsatisfactory due to non-unitary construction which can result in situ disintegration of the device and possible aspiration of a fragment of the device. The above devices do not have or teach the structural and functional features of the present invention described below which, in combination, enable the device to perform the intended function in a manner which is superior to the prior art devices. In particular, none of the prior art nasal airway devices include means operable for indicating the presence of an airborne pathogen entering the nasal passages, or for delivering an aromatherapeutic composition to a person's air passages.

The potential for human exposure to dangerous concentrations of noxious airborne pathogens such as anthrax spores has increased dramatically in recent months due to the introduction of pathogenic organisms into the work environment by deliberate criminal intent. Exposure to airborne pathogens such as anthrax spores and sarin gas is highly probable in the future, particularly for members of the military engaged in hostile action. In the case of anthrax, it is imperative that detection means operable for early detection of such human exposure to the airborne pathogen be available to both civilian and military populations. Ideally, such a detection system provides an indication and measure of an individual's exposure to a particular pathogen so that only individuals who have received a dangerously high level of exposure receive preemptive treatment. Accordingly, a detection device adapted to be worn upon the body and disposed to sample air that is actually inhaled by an individual is particularly desirable.

Aromatherapy has become a popular means for delivering volatile compositions to the airway of a person in order to elicit a desired response. Aromatherapy is the practice of using volatile plant oils, including “essential oils”, for psychological and physical well-being. Essential oils, which are the pure “essence” of a plant, may provide both psychological and physical benefits when used correctly. There are over 90 essential oils. Essential oils that are inhaled into the lungs are believed to offer both psychological and physical benefits. Not only does the aroma of the natural essential oil stimulate the brain to trigger a reaction, but the natural constituents (naturally occurring chemicals) of the essential oil are drawn into the lungs and can also provide physical benefits. There is a need for a device that can fit within the nose of a person, thereafter the device efficiently releasing essential oils or other volatile aromatherapeutic compositions into the person's airstream.

### SUMMARY OF THE INVENTION

It is a primary object of this invention to provide an intranasal device for selectively removing a sample of targeted pathogenic microorganisms from air delivered to the respiration passages and providing an indication of exposure to the targeted pathogenic microorganisms in the respiratory air.

It is a further object of this invention to provide a device adapted to be comfortably attached to a person's nose, thereafter being operable for releasing aromatherapeutic compositions to the resperatory air for transport and delivery of the aromatherapeutic composition to respiratory passages of the person.

It is another object of this invention to provide a device meeting the above objectives and which includes a means for controlling and/or limiting the projection of the device into the nostrils while preventing the device from dislodging and being ejected from the nostril.

It is another object of this invention to provide a device which meets the objectives stated above and which can be easily and inexpensively manufactured, at least in part, by injection molding from an inexpensive hypoallergenic plastic composite, copolymer or elastomer-coated plastic in a variety of sizes.

The above objects and advantages of the present invention are accomplished by the present nasal airway airborne pathogen indicator device. In a particularly preferred embodiment of an airborne pathogen indicator device in accordance with the present invention, the proximal dilating portion of the device comprises two “U” shaped surfaces adapted to be inserted into the nostrils. The dilating portion of the device is inter-connected by means of a bridging “U” shaped extension portion. The extension portion is a generally “U” shaped having a semicylindrical wall with an inner circumferential surface which is contoured to anatomically and snugly conform to the inferior (most distal) margin of the nasal septum. Thus, overall the device is generally “U” shaped, more or less resembling a cotter pin when viewed in front elevation and “U=U” shaped when the proximal portion is viewed end on from the top. The nasal airway dilating portion of the present device is described in detail in U.S. Pat. No. 5,931,852 to the present inventor. The bifurcated extension portion comprises two identical substantially planar parallel strips oriented with their flat surfaces in parallel planes and connected to one another by the semicylindrical distal septum attachment portion. The symmetrical dilating portion is contoured to anatomically conform to the respective contours of the anterior inner surface of the nostrils. Since inhaled air flowing through the nose contacts the inner surfaces of the “U's”, (i.e., the nontissue-contacting surfaces of the device), a pathogen-adsorbing coating, applied thereto, that is operable for removing at least a portion of the targeted pathogens, from the airflow, is employed to provide evidence of the wearer's exposure to selected, (targeted) airborne pathogens.

In an aromatheraputic composition delivery device of the present invention, one or more aromatherapeutic composition reservoir(s) are applied to the inner, nontissue-contacting surface(s) of the “U's”. When inhaled air flowing through the nose passes over the reservoir(s), the volatile aromatherapeutic composition is released into the airflow, thereafter to be transported into the person's respiratory passages where it passes into the bloodstream to elicit a desired response.

The features of the invention believed to be novel are set forth with particularity in the appended claims. However, the invention itself, both as to organization and method of operation, together with further objects and advantages thereof may best be understood by reference to the following description of a preferred embodiment of the invention, taken in conjunction with the accompanying drawings in which:

### BRIEF DESCRIPTION OF THE DRAWINGS

FIG. 1 is a front elevational view of a particularly preferred embodiment of an airborne pathogen indicating device in accordance with the present invention.

FIG. 2 is a perspective view of the airborne pathogen indicating device shown in FIG. 1.

FIG. 3 is a side view of the airborne pathogen indicating device of FIG. 2 viewed in the direction of line 3-3′.

FIG. 4 is a perspective inferior view of the airborne pathogen indicating device in accordance with the present invention positioned within the nose of a person and illustrating the disposition of the pathogen-sensitive coating with respect to the nasal airway passages.

FIG. 5 is a schematic lateral view of the airborne pathogen indicating device positioned within the nose which has been partially cut-a-way for illustration.

FIG. 6 is a perspective view of an embodiment of the invention having gripping pins adapted for facilitating instrument assisted insertion of the airborne pathogen indicating device into the nose.

FIG. 7 shows the cooperative functional relationship between the insertion instrument and the airborne pathogen indicating device of FIG. 6 prior to insertion of the device into the nose.

FIG. 8 is an elevational view of the airborne pathogen indicating device of FIG. 6 looking upward into the nose with the device properly positioned for operation and illustrating the trapping of particulate pathogens in the air by a cilia-like coating on a portion of the device.

FIG. 9 is a perspective view of an aromatherapeutic composition delivery embodiment of the invention having a pair of aromatherapeutic composition reservoirs affixed to the inner surfaces of the “U”-shaped members.

FIG. 10 is an elevational view of the aromatherapeutic composition delivery device of FIG. 9 looking upward into the nose with the device properly positioned for operation and illustrating the release of the volatile aromatherapeutic composition from the reservoirs affixed to the inner surface of the “U”-shaped members of the device.

### DESCRIPTION OF THE PREFERRED EMBODIMENTS

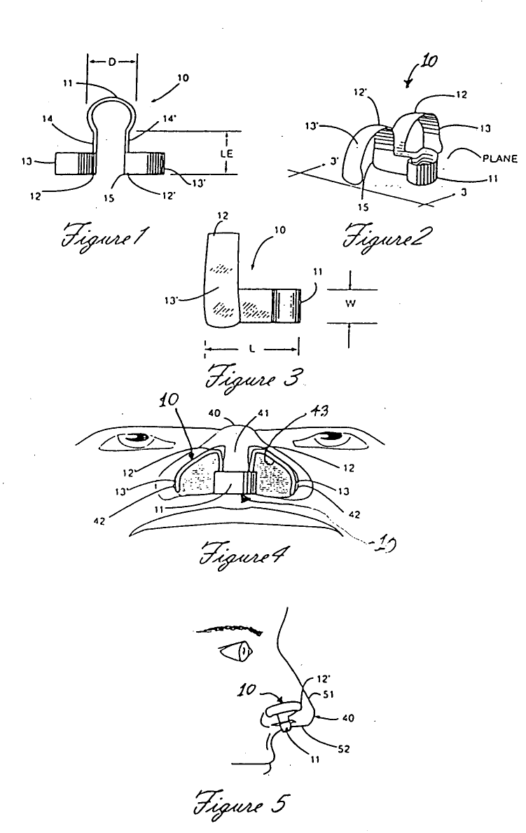

The airborne pathogen indicating device 10, shown in front (anterior) elevational view in FIG. 1, is a unitary strip of elastically deformable material, preferably hypoallergenic, elastomer shaped to form a symmetrical 3-dimensional structure as generally shown in FIG. 2. The airborne pathogen indicating device 10 is bifurcated and symmetrically disposed in structure with respect to the medial septum attachment portion 11. The septum attachment portion 11 is a semi-cylindrical arcuate distal coterminous for the inferior or distal end of the extension portion 14 and 14′ of the device 10, connecting the tines 14 and 14′ of the extension portion to one another and having an inner diameter D. The extension portion consists of tines 14 and 14′ and extends proximally, away from the septum attachment portion a distance L, the proximal terminus of the extension portions 14, 14′ being shown at 15. The dilating portion consists of two “U” shaped projections projecting anteriorly on a plane at right angles to the extension portion 14, to gracefully and arcuately curve laterally outward and posteriorly to form smooth, anatomically conforming tissue-contacting surfaces 13 and 13′ which are mirror images of each other. The side of the dilating portions opposite the tissue contacting surfaces 13 and 13′ are coated with a material that interacts with airborne pathogens in such a way as to enable the device to be used for determining exposure to one or more airborne pathogens. The extension portion 14 and 14′ preferably be a length L between 1/2 inch and 1 inch. The width W of the device 10 is substantially uniform throughout the device and preferably in the range of ⅛- 0.5/16 inches. The thickness of the device, excluding the thickness of the pathogen adsorbing coating 43, is also substantially uniform throughout and preferably less than 1/16 of an inch.

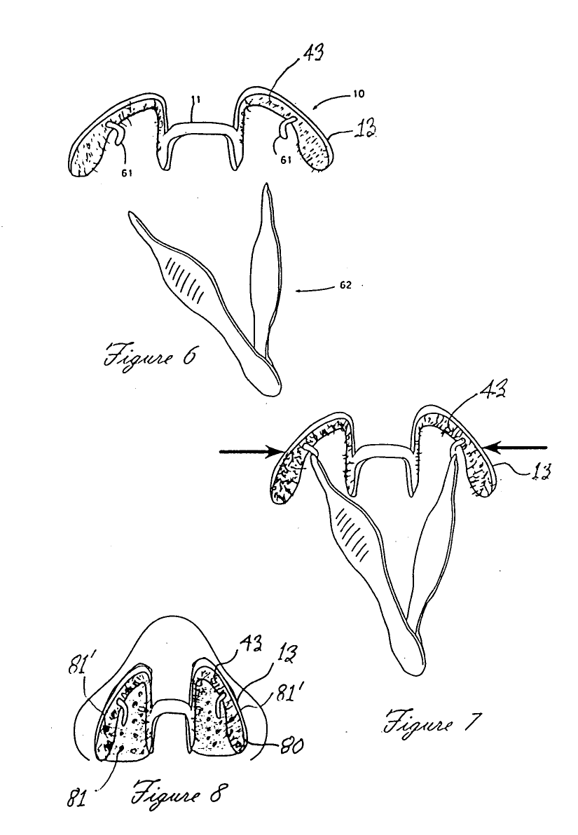

The device may be positioned within the nose 40 (FIG. 4) by inserting the proximal end 15 of the airborne pathogen indicating device 10 into the nasal air passages 42 and advanced by applying pressure on the distal opposing end until the distal septum attachment portion 11 is in contact with the inferior margin of the nasal septum 41. At this point, the proximal end 15 of the device 10 can be advanced no further into the nasal passages 42 and the anatomically conforming tissue-contacting surfaces 13 and 13′ comprising the dilating portion press laterally against the anterior and lateral wall of the nasal passage to dilate the passage and maintain an airway therethrough. A pathogen interactive coating 43 disposed on the inner surface (i.e., the surfaces opposite the tissue-contacting surfaces 13 and 13′) of the dilating portion of the airborne pathogen indicating device 10 interacts with airborne pathogens in such a way as to provide an indication that a pathogen is, or was, present in the airflow stream during periods of use. A coating 43 may, for example, have numerous cilia-like filaments extending into the airstream. Such a filamentous coating 43 (FIGS. 6-8) extracts pathogenic microorganisms from the airstream and concentrates the pathogens on an adherent coating on the filaments. The coating 43 may, for example, be a material that releases the pathogen accumulated thereon for assaying or other investigative purposes after treatment with an appropriate reagent, or provide a calorimetric indication of the presence of a particular airborne pathogen in contact therewith either with or without treatment by suitable indicator reagents.

The above-described embodiment is shown in FIG. 5 wherein the airborne pathogen indicating device 10 is seen to be comfortably positioned within the (partially sectioned) nose 40 of the patient. The surfaces 13 and 13′ elastically urge outward to press laterally outward against the wall of the nasal passage and provide a smooth, non-irritating tissue-contacting surface for comfort. The anterior projection 12′ of the dilating portion is shown facing the front or anterior portion 51 of the nose 40 and the septum attachment portion 11 is releasably attached to the inferior margin 41 of the nasal septum by medially directed elastic restorative forces.

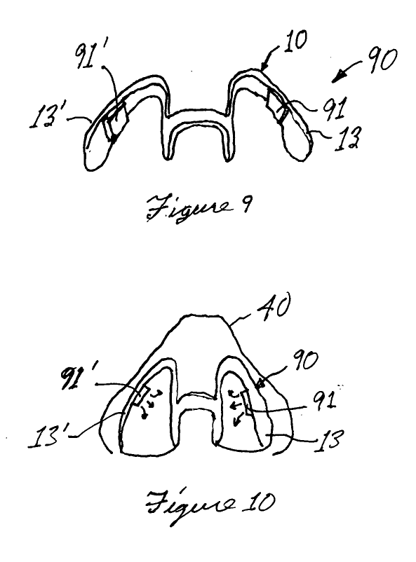

FIG. 6 is a perspective view of an embodiment of the airborne pathogen indicating device 10 including gripping pins 61 adapted to be grasped by an instrument 62 for facilitating instrument assisted insertion of the device 10 into the nose. The airborne pathogen interactive coating 43 is applied to a portion of the surface of the device 10 so as to contact air flowing through the nose. FIG. 7 shows the cooperative functional relationship between the insertion instrument 62 and device 10 prior to insertion of the device into the nose. The tines 14 and 14′ of the extension portion of the device are urged toward one another in the direction indicated by the broad arrows in FIG. 7 and the proximal end 15 inserted into the person's anterior nasal passages and advanced thereinto by means of the instrument 62. When the pressure exerted by the instrument 62 on the pins 61 is released, the tines of the extension portion bear against the lateral tissue surfaces of the medial nasal septum to stabilize the device while the proximal arcuate tissue-contacting surfaces of the dilating portion buttress a portion of the inner perimeter of the nasal passages, urging the tissue in contact therewith radially outward to create and maintain symmetrically disposed open nasal airways.

FIG. 8 is an elevational view looking upward into the nose 40 showing the airborne pathogen indicating device 10 properly positioned in accordance with this invention for maintaining an open airway within the nose, while presenting an interactive surface 43 to adsorb airborne pathogens 81 entrained in the air stream passing thereover. The airborne pathogen interactive coating 43, which may be a pathogen-specific adsorptive or reactive coating, may be affixed to the outer surface 80 (i.e., the surface in opposition to tissue contacting surface 13 and 13′) of the dilating portion of the airborne pathogen indicating device 10 to adsorb or chemically react with airborne pathogens 81 entrained in the air stream. FIG. 8 shows a portion 81′ of the airborne pathogens 81 trapped in a hydrophilic or hydrophobic gel coating applied to cilia-like fibrils 43. In addition to the present invention's utility for maintaining an airway and indicating exposure to an airborne pathogen, the tissue-contacting surfaces 13 and 13′ of the device may be open cell or porous and permeated with a medicament that is released into the tissue in contact therewith.

FIG. 9 is a bottom perspective view of an aromatherapeutic composition delivery embodiment of the invention having at least one, and more preferably a pair of aromatherapeutic composition reservoirs 91 and 91′ affixed to the inner (nontissue-contacting) surfaces of the “U”-shaped members 13 and 13′ of the airway device 10. The reservoirs 91 and 91′ are operable for storing an aromatherapeutic composition in liquid phase such as a volatile essential oil, and releasing only essential oil vapor into the airstream. The reservoirs, which may be a fabric such as felt or a sponge, preferably have a large surface area capable of adsorbing the liquid phase of the aromatherapeutic composition and releasing the vapor phase of the adsorbate into air. The aromatherapeutic composition stored in the reservoirs 91 and 91′ may be replenished when it becomes depleted. FIG. 10 is an elevational view of the aromatherapeutic composition delivery device 90 of FIG. 9 looking upward into the nose 92 with the device properly positioned for operation and illustrating the release (indicated by small arrows) of the volatile aromatherapeutic composition from the reservoirs 91 and 91′ affixed to the inner surface of the “U”-shaped members of the device 90.

With reference to FIGS. 6-8, a preferred method for providing a pathogen-adsorbing coating 43 comprises the application of a thin film of an adsorbant or chemically reactive reagent such as a hydrophilic hydrogel or a hydrophobic silicone gel coating containing an indicator reagent that provides, or can be further treated to provide, an indication of exposure to an airborne pathogen to the surface 80 of the airborne pathogen indicating device 10. After use, the coated surface 43 of the device can be either visually inspected to indicate exposure to an airborne pathogen, or the coating may be further treated by a visualizing reagent as, for example, in a home test kit, to provide an indication of the exposure of the coated surface 43 to a targeted airborne pathogen. In a particularly preferred embodiment, the coating 43 comprises a layer of synthetic polymer having fibrils or a tortuous surface projecting into the air stream. The fibrils are coated with either a hydrophilic hydrogel or a hydrophobic silicone gel, the choice of gel coating depending on the hydrophilicity of the cell wall of the pathogen targeted for detection. Pathogenic bacteria such as Bacillis anthracis, are characterized, at least in part, by having a cell wall. The outer surface of the cell wall comprises lipids and proteins. The cell wall can be either hydrophobic or hydrophilic, depending on structure and distribution of the lipids and proteins within the outer surface of the cell wall of the organism. If the fibers are coated with a hydrophilic material such as a hydrogel (eg., polyvinylpyrrolidone), organisms having a hydrophilic cell wall will be adsorbed and adhere to the coating as air passes over the fibers whereas organisms having a hydrophobic cell wall will have little or no affinity for the coating. Conversely, if the fibers are coated with a hydrophobic material such as silicone gel, organisms having a hydrophobic cell wall will adhere. After use, at the end of a day, for example, the device 10 can be removed from the nose and treated by immersing the coating 43 in a solvent that will dissolve the coating and/or the fibrils and release the pathogenic organsims, entrapped and accumulated therein, into the solvent. The solvent containing the pathogenic organisms in suspension can be mounted on a slide for microscopic examination, cultured, or otherwise analyzed for detection of the presence of, and the determination of the amount of the pathogenic organism, such as, for example, anthrax spores, that may be injurious to health. In addition, a test kit may be used in a home environment for providing a visual indication, such as a color change, of exposure of the coating 43 to an airborne pathogen. Appropriate medicaments such as, for example, antibiotics exhibiting a therapeutic effect, may then be timely administered to the patient if a high level of exposure to a particular pathogen is indicated.

While particular embodiments of the present invention have been illustrated and described, it would be obvious to those skilled in the art that various other changes and modifications can be made without departing from the spirit and scope of the invention. For example, the airborne pathogen indicating device 10 is applicable, or may be adapted to concentrate and/or detect any number of different airborne pathogens, the particular pathogens detected depending on the specificity of the coating 43. Further, while a particular embodiment of an airway device that is suitable for supporting a pathogen sensitive or adsorbant coating within the nose and present the coating to the air stream passing through the nose, has been described, the particular device 10 is intended to provide a preferred exemplar of the invention. Many other possible geometries are possible for the airborne pathogen indicating device 10. For example, a single length of elastomeric tubing or a spring-loaded semi or hemi tube dimensioned to fit snugly within the nose and present a reactive surface to the air stream may be used to meet the objectives of the present invention. As a further example, persons such as coroners engaged in the practice of forensic science are subjected to abhorent odors such as decomposing human tissue. It is desireable to mask these objectionable odors with a more intense pleasant scent. The aromatherapeutic composition delivery device of the present invention may be used to deliver masking scents to the airstream. It is therefore intended to cover in the appended claims all such changes and modifications that are within the scope of this invention.

1. An airborne pathogen indicating device adapted for attachment to a person's nose for indicating the presence of a microbial pathogen entrained in an air stream passing into the nose of an animal, said airborne contaminant indicating device comprising a member dimensioned to fit snugly within a nose without substantially obstructing the flow of air through the nose, said member having a surface that is in direct contact with air passing through the nose, said surface comprising a gel operable for selectively adsorbing said microbial pathogen.

2. An airborne pathogen indicating device adapted for attachment to a person's nose for indicating the presence of a microbial pathogen entrained in an air stream passing into the nasal passage of the nose, said airborne pathogen indicating device comprising:
(a) a dilating portion comprising two substantially U-shaped strips of an elastically deformable biocompatible material, said substantially U-shaped strips lying substantially in a first plane and being mirror images of one another and having a substantially uniform width and a smooth outer, tissue-contacting surface and an inner air-contacting surface in opposition thereto; and
(b) an extension portion integral with said dilating portion comprising two straight strips of said elastically deformable biocompatible material having a length and said substantially uniform width, each of said two straight strips having a proximal end integral with one of the two U-shaped strips, a distal end, and an outer septum-contacting surface which is coplanar with at least a portion of said tissue-contacting surface of said U-shaped strip integral therewith; and
(c) a septum attachment portion comprising a substantially arcuate strip of said elastically deformable material having said substantially uniform width, said substantially arcuate strip having two parallel straight edges, each straight edge being integral with a distal end of one of said straight strips comprising the extension portion wherein said two straight strips and said arcuate strip lie in a second plane which is orthogonal to said first plane; and
(d) a coating on said outer surface of said dilating portion, operable for adsorbing said airborne pathogenic organism.

3. (canceled)

4. (canceled)

5. The airborne pathogen indicating device of claim 1 wherein said coating on said outer surface comprises a hydrophilic gel.

6. The airborne pathogen indicating device of claim 1 wherein said coating on said outer surface comprises a hydrophobic gel.

7. The airborne pathogen indicating device of claim 2 wherein said coating on said outer surface comprises a hydrophilic gel.

8. The airborne pathogen indicating device of claim 2 wherein said coating on said outer surface comprises a hydrophobic gel.

9. An aromatherapy device adapted for attachment to a person's nose for dispensing a volatile aromatherapeutic composition into an air stream passing into the nose of the person, said aromatherapy device comprising a member dimensioned to fit snugly within a nose without substantially obstructing the flow of air through the nose, said member having a surface that is in direct contact with air passing through the nose, said surface having at least one aromatherapeutic composition reservoir disposed thereon operable for storing a liquid phase of a volatile aromatherapeutic composition therein and releasing a vapor phase of said aromatherapeutic composition into air passing into the nose.

10. The aromatherapy device of claim 9 wherein said aromatherapeutic composition comprises a volatile component providing a masking scent.
